(19) United States
(12) Patent Application Publication
(10) Pub. No.: US 2019/0365940 A1
(43) Pub. Date: Dec. 5, 2019

(54) SCENTED PACKAGING FOR AN APPLIANCE

(71) Applicant: Haier US Appliance Solutions, Inc., Wilmington, DE (US)

(72) Inventors: Alexander B. Leibman, Prospect, KY (US); Michael Glenn Mattingly, Louisville, KY (US); Zackary Filbert, Louisville, KY (US)

(21) Appl. No.: 15/992,297

(22) Filed: May 30, 2018

(51) Int. Cl. A61L 9/04; D06F 39/12; C09J 11/00; C09J 7/00

(52) U.S. Cl. CPC A61L 9/042; D06F 39/12; A61L 9/04; C09J 7/00; C09J 11/00

(57) ABSTRACT

An appliance having one or more scented packaging materials is provided. The scented packaging materials removably attach to or support the appliance, e.g., during shipping. A preselected scent emanates from the scented packaging materials. Thus, when the appliance is delivered to a consumer, the preselected scent emanating from the scented packaging materials provides the appliance with an attractive smell.

### FIELD OF THE INVENTION

The present subject matter relates generally to consumer appliances and more particularly to scented packaging for such appliances.

### BACKGROUND OF THE INVENTION

When consumer appliances are delivered to consumers, the smell or sent of the appliance may be less than desirable or unpleasant to consumers. The appliance itself and the packaging materials attached to or supporting the appliance during shipping may have unpleasant odors or simply no distinctive smell. Thus, at the outset, consumers may associate their appliance with an unpleasant smell or fail to associate any smell with their appliance.

Accordingly, an appliance that includes scented packaging that is removably attached to or that supports the appliance, e.g., during shipping, that addresses one or more of the challenges noted above would be desirable.

### BRIEF DESCRIPTION OF THE INVENTION

Aspects and advantages of the invention will be set forth in part in the following description, or may be apparent from the description, or may be learned through practice of the invention.

In one exemplary embodiment, an appliance is provided. The appliance includes a housing defining an interior volume. The appliance also includes a door coupled with the housing for selectively providing access to the interior volume of the appliance. Further, the appliance includes a scented packaging material removably attached to or supporting at least one of the housing and the door, wherein a preselected scent emanates from the scented packaging material.

In another exemplary embodiment, an appliance is provided. The appliance includes a housing defining an interior volume. The appliance also includes a door coupled with the housing and movable between an open position and a closed position for selectively providing access to the interior volume of the appliance. Further, the appliance includes one or more scented packaging materials removably attached to or supporting at least one of the housing and the door, wherein a preselected scent emanates from the one or more scented packaging materials, and wherein at least one of the one or more scented packaging materials is removably disposed within the interior volume of the housing, and wherein when the door is moved to the open position, the preselected scent emanating from the scented packaging materials removably disposed within the interior volume emanates to an ambient environment surrounding the appliance.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 provides a perspective view of a washing machine appliance according to an exemplary embodiment of the present disclosure with a door of the exemplary washing machine appliance shown in a closed position;

FIG. 2 provides a perspective view of the exemplary washing machine appliance of FIG. 1 with the door of the washing machine appliance shown in an open position;

FIG. 3 provides a side cross-sectional view of the washing machine appliance of FIG. 1;

FIG. 4 provides a top, perspective view of an exemplary washing machine appliance having various scented packaging materials attached to and supporting the washing machine appliance according to an exemplary embodiment of the present disclosure;

FIG. 5 provides a schematic view of an exemplary scented tape used for packaging the appliance according to an exemplary embodiment of the present disclosure;

FIG. 6 provides a schematic view of an exemplary scented tape used for packaging the appliance according to an exemplary embodiment of the present disclosure;

FIG. 7 provides a schematic view of an exemplary scented plastic sheet used for packaging the appliance according to an exemplary embodiment of the present disclosure;

FIG. 8 provides a close up, perspective view of an exemplary washing machine appliance depicting a door of the washing machine appliance in an open position to reveal various bags that may be disposed within an interior volume of the washing machine appliance according to an exemplary embodiment of the present disclosure; and

FIG. 9 provides a perspective view of an exemplary appliance packaged within a box and with expanded polystyrene support members supporting the appliance according to an exemplary embodiment of the present disclosure.

Repeat use of reference characters in the present specification and drawings is intended to represent the same or analogous features or elements of the present invention.

### DETAILED DESCRIPTION OF THE INVENTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents. As used herein, terms of approximation, such as “approximately,” “substantially,” or “about,” refer to being within a ten percent margin of error.

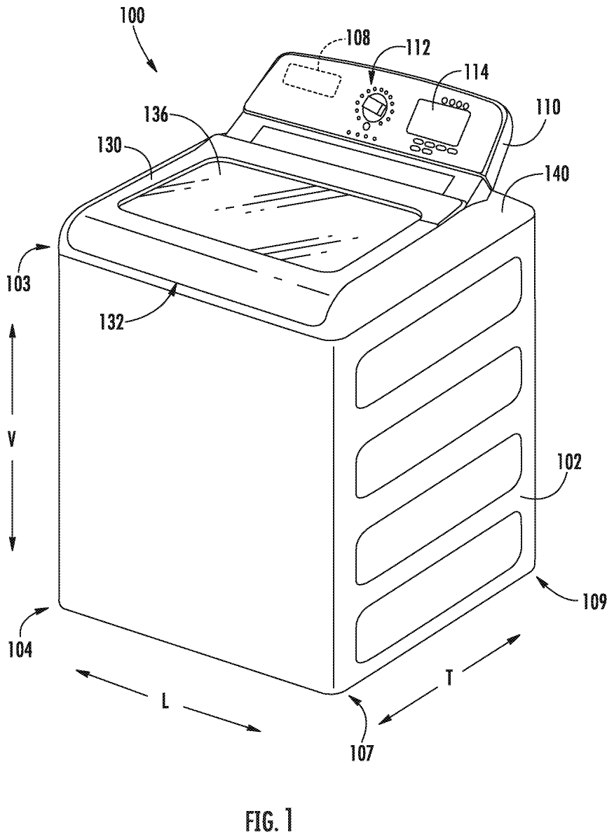

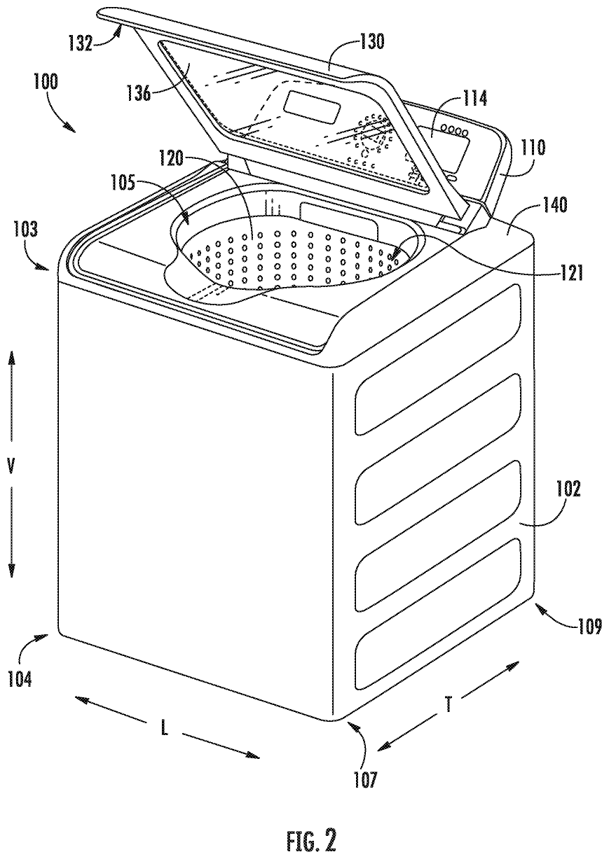

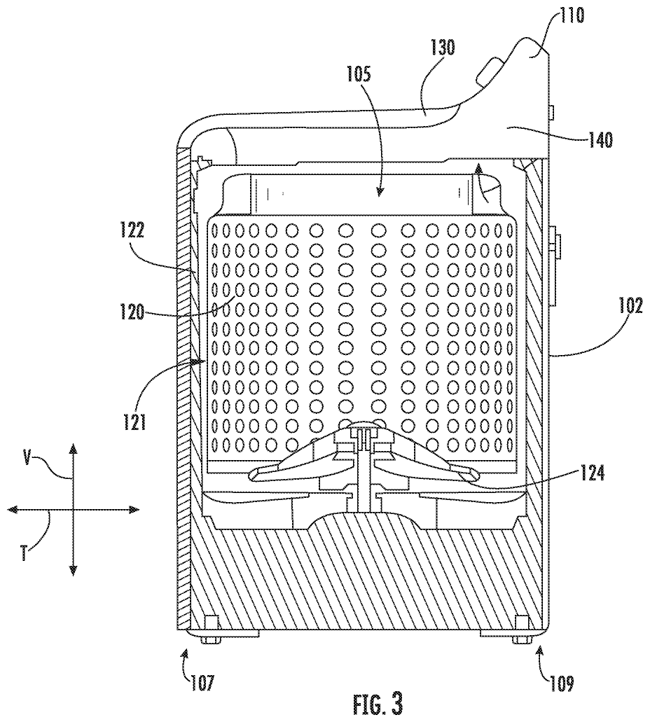

FIGS. 1, 2, and 3 illustrate one exemplary appliance in which aspects of the present disclosure may be incorporated. In particular, FIGS. 1 through 3 provide an exemplary vertical axis washing machine appliance 100. In FIG. 1, a lid or door 130 of washing machine appliance 100 is shown in a closed position. In FIG. 2, door 130 is shown in an open position. FIG. 3 provides a side cross-sectional view of the washing machine appliance of FIGS. 1 and 2. As shown in FIGS. 1 and 2, washing machine appliance 100 defines a vertical direction V, a lateral direction L, and a transverse direction T, each of which is mutually perpendicular such that an orthogonal coordinate system is defined.

While described in the context of a specific embodiment of vertical axis washing machine appliance 100, using the teachings disclosed herein it will be understood that vertical axis washing machine appliance 100 is provided by way of example only. Other washing machine appliances having different configurations, different appearances, and/or different features may also be utilized with the teachings of the present disclosure as well, e.g., horizontal axis washing machines. Further, as will be described herein, the teachings of the present disclosure are applicable and may be incorporated in or with other types of appliances, such as e.g., a dryer appliance, a microwave appliance, a refrigerator appliance, a toaster oven appliance, and oven appliance, a range appliance, etc.

With reference to FIGS. 1 and 2, washing machine appliance 100 has a cabinet or housing 102 that extends between a top portion 103 and a bottom portion 104 along the vertical direction V and between a front 107 and a back 109 along the transverse direction T. A wash basket 120 (FIGS. 2 and 3) is rotatably mounted within housing 102. A motor (not shown) is in mechanical communication with wash basket 120 to selectively rotate wash basket 120 (e.g., during an agitation or a rinse cycle of washing machine appliance 100). Wash basket 120 is received within an interior volume or wash chamber 121 (FIGS. 2 and 3) defined by a tub 122 (FIG. 3) positioned within housing 102. Wash basket 120 is configured for receipt of articles for washing. Tub 122 holds wash and rinse fluids for agitation in wash basket 120 within chamber 121. An agitator or impeller 124 extends into wash basket 120 and is also in mechanical communication with the motor. The impeller 124 facilitates agitation of articles disposed within wash basket 120 during operation of washing machine appliance 100.

Housing 102 of washing machine appliance 100 has a top panel 140. Top panel 140 defines an opening 105 (FIG. 2) that permits user access to wash basket 120 of chamber 121. Door 130, rotatably mounted to top panel 140, permits selective access to opening 105; in particular, door 130 selectively rotates or is movable between the closed position shown in FIG. 1 and the open position shown in FIG. 2. In the closed position, door 130 inhibits access to wash basket 120. Conversely, in the open position, a user can access wash basket 120. A window 136 in door 130 permits viewing of wash basket 120 when door 130 is in the closed position, e.g., during operation of washing machine appliance 100. Door 130 also includes a handle 132 that, e.g., a user may pull and/or lift when opening and closing door 130. Further, although door 130 is illustrated as mounted to top panel 140, alternatively, door 130 may be mounted to housing 102 or any other suitable support member.

A control panel 110 with at least one input selector 112 (FIG. 1) extends from top panel 140. Control panel 110 and input selector 112 collectively form a user interface for operator selection of machine cycles and features. A display 114 of control panel 110 indicates selected features, operation mode, a countdown timer, and/or other items of interest to appliance users regarding operation.

Operation of washing machine appliance 100 is controlled by a controller or processing device 108 (FIG. 1) that is operatively coupled to control panel 110 for user manipulation to select washing machine cycles and features. In response to user manipulation of control panel 110, controller 108 operates the various components of washing machine appliance 100 to execute selected machine cycles and features.

Controller 108 may include a memory and microprocessor, such as a general or special purpose microprocessor operable to execute programming instructions or micro-control code associated with a cleaning cycle. The memory may represent random access memory such as DRAM, or read only memory such as ROM or FLASH. In one embodiment, the processor executes programming instructions stored in memory. The memory may be a separate component from the processor or may be included onboard within the processor. Alternatively, controller 108 may be constructed without using a microprocessor, e.g., using a combination of discrete analog and/or digital logic circuitry (such as switches, amplifiers, integrators, comparators, flip-flops, AND gates, and the like) to perform control functionality instead of relying upon software. Control panel 110 and other components of washing machine appliance 100 may be in communication with controller 108 via one or more signal lines or shared communication busses.

During operation of washing machine appliance 100, laundry items are loaded into wash basket 120 through opening 105, and washing operation is initiated through operator manipulation of input selectors 112. Wash basket 120 is filled with water and detergent and/or other fluid additives. One or more valves can be controlled by washing machine appliance 100 to provide for filling wash basket 120 to the appropriate level for the amount of articles being washed and/or rinsed. By way of example for a wash mode, once wash basket 120 is properly filled with fluid, the contents of wash basket 120 can be agitated (e.g., with an impeller as discussed previously) for washing of laundry items in wash basket 120.

After the agitation phase of the wash cycle is completed, wash basket 120 can be drained. Laundry articles can then be rinsed by again adding fluid to wash basket 120 depending on the specifics of the cleaning cycle selected by a user. The impeller may again provide agitation within wash basket 120. One or more spin cycles also may be used. In particular, a spin cycle may be applied after the wash cycle and/or after the rinse cycle to wring wash fluid from the articles being washed. During a spin cycle, wash basket 120 is rotated at relatively high speeds. After articles disposed in wash basket 120 are cleaned and/or washed, the user can remove the articles from wash basket 120, e.g., by reaching into wash basket 120 through opening 105.

Washing machine appliance 100 may be packaged with and/or contained within various packaging materials, e.g., to protect dishwasher appliance 100 from damage during shipping. For instance, surfaces of housing 102, door 130, and various other components or surfaces of washing machine appliance 100 may be protected with plastic sheets. Further, numerous pieces of tape may be attached to the washing machine appliance 100. The tape may be attached directly to surfaces of washing machine appliance 100 or may be attached indirectly, e.g., to a plastic sheet covering one of the surfaces of washing machine appliance 100. For instance, tape may be used to keep or retain door 130 in the closed position during shipping, e.g., by adhering to door 130 and a portion of the housing 102. Moreover, various packaging materials may be disposed within the interior volume or wash chamber 121 of washing machine appliance 100, e.g., to secure wash basket 120 in place during shipping. In addition, various bags may be disposed within wash basket 120 as well. For example, an owner's manual may be contained within a bag disposed within wash basket 120. As another example, a bag containing various starter detergents, wash additives, and/or soaps may be disposed within wash basket 120. Furthermore, packaging materials may include expanded polystyrene (EPS) material positioned surrounding the washing machine appliance 100. For instance, an EPS packaging support member may be positioned at top 103 and/or bottom 104 of housing 102, e.g., to support washing machine appliance 100 during shipping. Packaging materials may also include a box or other shipping container. Washing machine appliance 100 may be contained within the box or shipping container, e.g., for ease of handling and damage prevention. In accordance with exemplary aspects of the present disclosure, one or more of such packaging materials that removably attach to or support washing machine appliance 100, e.g., during storage and shipping, may be scented packaging materials. A preselected scent emanates from the one or more scented packaging materials. In this way, when washing machine appliance 100 or an appliance packaged in such scented packaging materials is delivered to a consumer, the preselected scent of the packaging materials provides the appliance with an attractive smell. Thus, a user may associate his or her new appliance with the attractive smell.

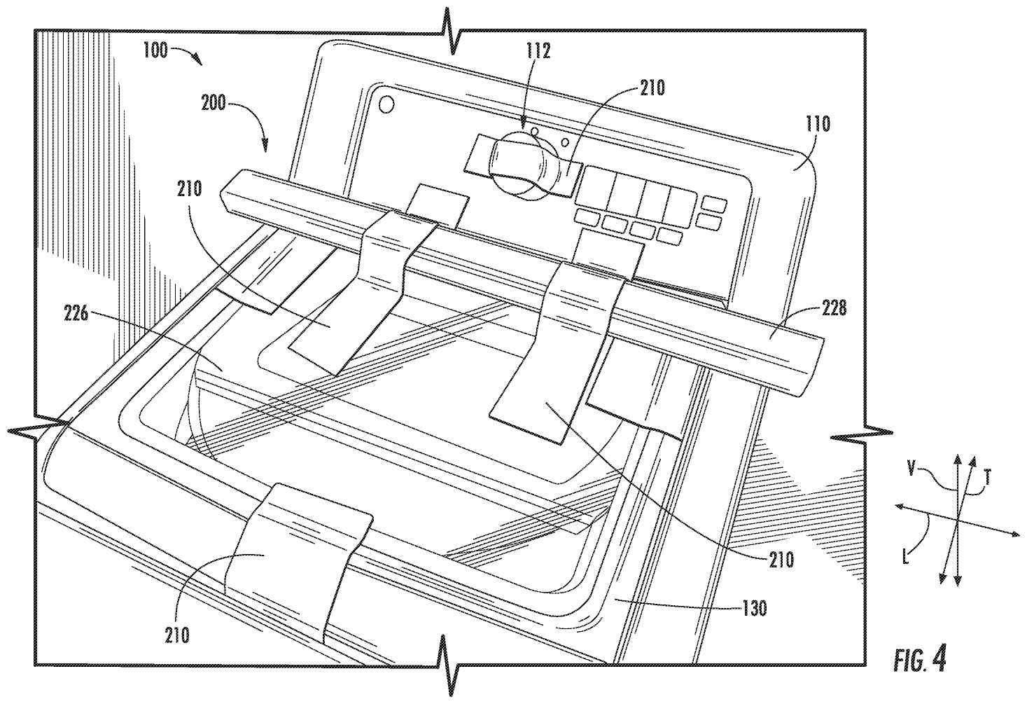

FIG. 4 provides a top, perspective view of an appliance having various scented packaging materials attached to and/or supporting the appliance according to an exemplary embodiment of the present disclosure. For this embodiment, the appliance is the washing machine appliance 100 of FIGS. 1 through 3. As shown, washing machine appliance 100 includes scented packaging materials 200 removably attached to or supporting the appliance, e.g., attached to housing 102 or supporting at least one of the housing 102 and door 130. In particular, the scented packaging materials 200 removably attached to washing machine appliance 100 includes tape 210, a plastic sheet 226 attached to door 130, and a retention bar 228 positioned proximate the hinge (not shown) of door 130 and extending along the length of washing machine appliance 100 along the lateral direction L. Retention bar 228, along with tape 210 and plastic sheet 226, are configured to retain door 130 in the closed position, e.g., during shipping, and also to protect door 130.

A preselected scent emanates from the scented packaging materials 200 depicted in FIG. 4. That is, a preselected scent emanates from tape 210, plastic sheet 226, and retention bar 228. The preselected scent may be applied to the packaging materials in any suitable manner. As one example, the scented packaging materials 200 may be soaked or quenched in a bath or vessel containing a scented liquid during manufacturing. The chemical compound(s) of the scented liquid may absorb into the packaging material and thus the packaging material may become scented. As another example, packaging materials may be sprayed with the desired scent such that the packaging material absorbs the chemical compound of the desired scent. As yet another example, the chemical compounds of the desired smell may be formulated with the packaging material, e.g., the chemicals of the selected scent may be formulated with an adhesive layer of tape 210. Other suitable methods are possible.

The preselected scent may be any suitable scent. For instance, example preselected scents may have a citrus scent, such as orange, citron, lemon, lime, orange, grapefruit, some combination thereof. As another example, the preselected scent may have a floral scent, such as lavender, jasmine, rosemary, other floral scent, or some combination thereof. As yet another example, the preselected scent may have a spice scent, such as a peppermint, vanilla, or cinnamon smell. As further examples, the preselected scent may include one or more aroma compounds, including various esters, linear terpenes, cyclic terpenes, and aromatics. Example ester compounds include: geranyl acetate, methyl formate, methyl acetate, methyl propionate, methyl propanoate, methyl butyrate, methyl butanoate, ethyl acetate, ethyl butyrate, ethyl butanoate, isoamyl acetate, pentyl butyrate, pentyl butanoate, pentyl pentanoate, octyl acetate, benzyl acetate, and methyl anthranilate. Example linear terpene compounds include: myrcene, geraniol, nerol, citral, lemonal geranial, neral, citronellal, citronellol, linalool, and nerolidol. Example cyclic terpene compounds include: limonene, camphor, menthol, carvone, terpineol, alpha-ionone, thuj one, and eucalyptol. Example aromatic compounds include: benzaldehyde, eugenol, cinnamaldehyde, ethyl maltol, vanillin, anisole, anethole, estragole, and thymol. In some embodiments, the preselected scent is perceptible to a human having a normal sense of smell. In some embodiments, the preselected scent is either consciously or unconsciously perceptible to a human. In some embodiments, the preselected scent is configured to target a specific olfactory gland of a human.

As depicted in FIG. 4 and as noted above, the scented packaging materials 200 removably attached to or supporting washing machine appliance 100 includes tape 210, or in this embodiment a plurality of tape pieces. Tape 210 is used to secure retention bar 228. As shown, tape 210 extends over retention bar 228 and connects door 130 with control panel 110. Thus, tape 210 is attached to at least one of housing 102 and door 130, and in this depicted embodiment, tape 210 is attached to both housing 102 and door 130. Tape 210 is used to cover and protect various components of washing machine appliance 100, such as e.g., input selector 112 as shown in FIG. 4.

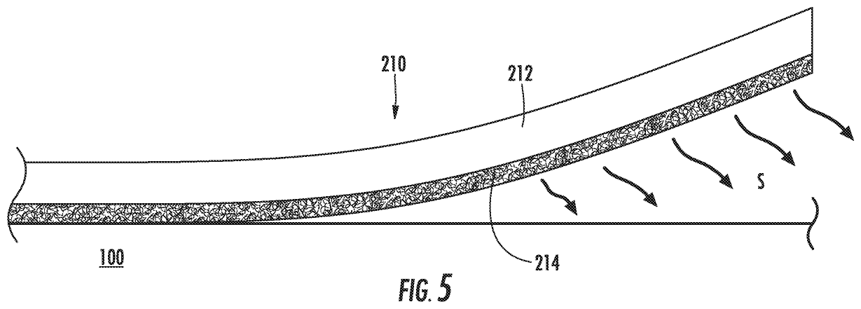

In some embodiments, the preselected scent emanates from the scented packaging material only upon removal of the packaging material from the appliance. By way of example, FIG. 5 provides one example embodiment of tape 210. For this embodiment, tape 210 includes a cover layer 212 and an adhesive layer 214. For this embodiment, adhesive layer 214 is formulated in such a way that is has a preselected scent S. In this way, when a consumer removes tape 210 from washing machine appliance 100, e.g., by peeling tape 210, adhesive layer 214 becomes exposed to the ambient air surrounding washing machine appliance 100. Consequently, the preselected scent S emanates from the adhesive layer 214. Accordingly, an attractive smell is delivered to the consumer upon removal of tape 210 from washing machine appliance 100. Moreover, as adhesive layer 214 of tape 210 is covered by cover layer 212 when tape 210 is attached to washing machine appliance 100 (i.e., adhesive layer 214 is not exposed to the ambient air when tape 210 is attached to the appliance), the preselected scent S does not prematurely emanate from the appliance, e.g., during shipping prior to delivery of the appliance to the consumer. Thus, the preselected scent S emanates from tape 210 only upon removal of tape 210 from the appliance.

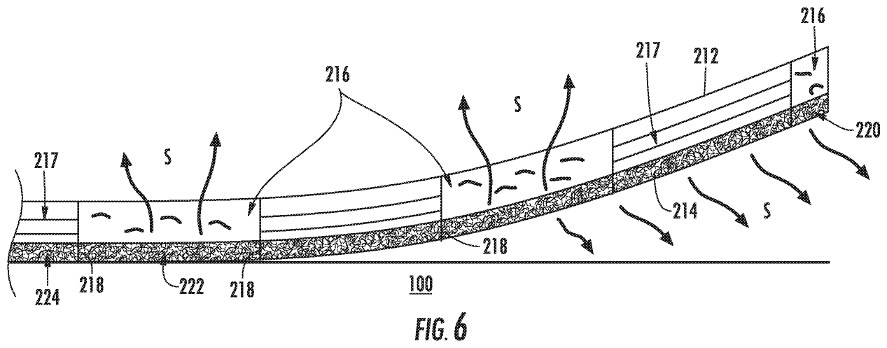

FIG. 6 provides another example embodiment of tape 210. For this embodiment, tape 210 includes a cover layer 212 and an adhesive layer 214. Cover layer 212 includes one or more porous sections 216 that allow the preselected scent S to vent or emanate therethrough even when tape 210 is attached to a surface of washing machine appliance 100. Accordingly, the preselected scent S of tape 210 may emanate from tape 210 when tape 210 is attached to one of the surfaces of washing machine appliance 100 or when removed from one of the surfaces of washing machine appliance 100. In this manner, tape 210 may provide an attractive smell to a consumer before tape 210 is removed from washing machine appliance 100 and after tape 210 has been removed.

To prevent the whole of the preselected scent S from emanating from tape 210 prior to delivery of washing machine appliance 100 to the consumer, e.g., in the event washing machine appliance 100 is delivered to a consumer after some time, tape 210 includes boundary walls 218 partitioning adhesive layer 214 into adhesive sections 220. As shown, at least one of the adhesive sections 220 is in fluid communication with one of the one or more porous sections 216 to allow the preselected scent S to emanate therethrough even when tape 210 is attached to at least one of housing 102 and door 130, or more generally, washing machine appliance 100. For instance, a first adhesive section 222 is in fluid communication with one of the porous sections 216 of cover layer 212. Further, as depicted, at least one of the adhesive sections 220 is sealed by a non-porous section 217 of cover layer 212, boundary walls 218, and at least one of housing 102 and door 130 when tape 210 is attached to at least one of housing 102 and door 130, or more generally, washing machine appliance 100. For instance, a second adhesive layer 224 is shown sealed by non-porous section 217 of cover layer 212, boundary walls 218, and at least one of housing 102 and door 130 when tape 210 is attached to washing machine appliance 100. Thus, even if the preselected scent S stemming from first adhesive section 222 completely dissipates or emanates to the ambient air prior to delivery of washing machine appliance 100 to a consumer, second adhesive section 224 of adhesive layer 214 may retain its preselected scent S until it is removed from washing machine appliance 100, e.g., so that the preselected scent S may be released when tape 210 is removed from washing machine appliance 100. Further, in some embodiments, additionally or alternatively to the embodiments noted above, cover layer 212 of tape 210 may likewise have a scented composition that emanates preselected scent S.

Returning to FIG. 4, as noted above for this embodiment, the scented packaging materials 200 removably attached to or supporting washing machine appliance 100 include plastic sheet 226 attached to door 130 and retention bar 228. Stated differently, plastic sheet 226 and retention bar 228 each have a scented composition that emanates the preselected scent S. In this way, the preselected scent S may emanate from plastic sheet 226 and retention bar 228 in addition to tape 210.

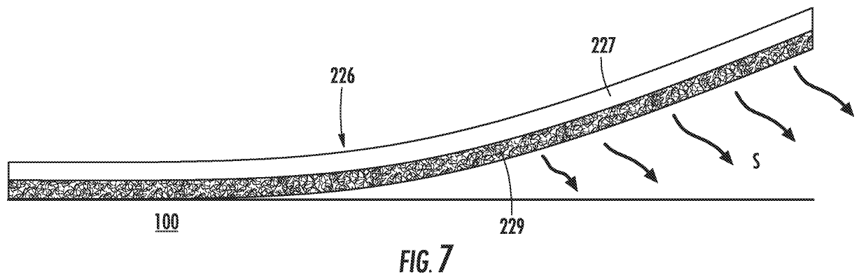

In some embodiments, one or more of the plastic sheets 226 covering washing machine appliance 100 may be structurally similar to tape 210 shown in FIG. 5. In such embodiments, as shown in FIG. 7, a representative plastic sheet 226 includes a protective film 227 and an adhesive layer 229. The adhesive layer 229 is formulated in such a way that is has a preselected scent S. In this way, when a consumer removes plastic sheet 226 from washing machine appliance 100, e.g., by peeling plastic sheet 226, adhesive layer 229 becomes exposed to the ambient air surrounding washing machine appliance 100. Consequently, the preselected scent S emanates from the adhesive layer 229. Accordingly, an attractive smell is delivered to the consumer upon removal of plastic sheet 226 from washing machine appliance 100. Moreover, as adhesive layer 229 of plastic sheet 226 is covered by protective film 227 when plastic sheet 226 is attached to washing machine appliance 100 (i.e., adhesive layer 229 is not exposed to the ambient air when plastic sheet 226 is attached to the appliance), the preselected scent S does not prematurely emanate from the appliance, e.g., during shipping prior to delivery of the appliance to the consumer. Thus, the preselected scent S emanates from plastic sheet 226 only upon removal of plastic sheet 226 from the appliance.

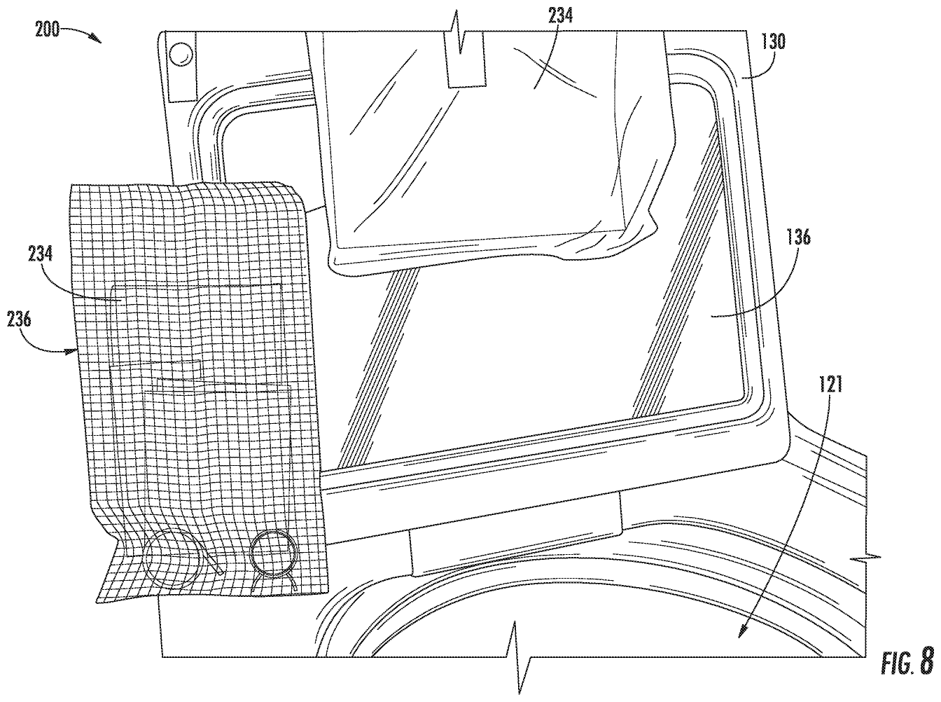

In some exemplary embodiments, various scented packaging materials are removably disposed within an interior volume of the appliance. In this manner, when the door of the appliance is moved to the open position, the preselected scent emanating from the scented packaging material emanates from the interior volume to an ambient environment surrounding the appliance. Accordingly, a wave of attractive smells may be delivered to the consumer. For instance, as shown in FIG. 4, the scented packaging material 200 disposed within the interior volume or wash chamber 121 is an expanded polystyrene (EPS) material 230 positioned to secure wash basket 120 in place. That is, the EPS material 230 prevents wash basket 120 from rotating about its axis of rotation and translating or rotating along one of the directions of the orthogonal direction system. When EPS material 230 is positioned within wash chamber 121 and door 130 is closed and secured as shown in FIG. 4 (e.g., at the factory), the preselected scent of EPS material 230 emanates therefrom and mixes with the air within wash chamber 121. When a consumer opens door 130, e.g., by removing tape 210, plastic sheet 226, and retention bar 228 and rotating door 130 to the open position (as shown in FIG. 8), the preselected scent S “bottled up” or filling the interior volume or wash chamber 121 of washing machine appliance 100 dissipates into the surrounding ambient air thus delivering an attractive wave of smell to the consumer. Once the consumer smells the attractive smell, the consumer may associate the appliance with the attractive smell.

FIG. 8 provides a close up, perspective view of washing machine appliance 100 of FIG. 4 depicting door 130 in an open position. As shown, in some embodiments, other scented packaging materials 200 that may be removably disposed within the interior volume of the appliance include various bags. For this embodiment, a documentation bag 232 is shown attached to a bottom surface of door 130. The documentation bag 232 may contain various documents associated with washing machine appliance 100, such as e.g., an owner's manual and assembly instructions. Another bag that may be removably disposed within the interior volume of washing machine appliance 100 is a treatment bag 234 that includes various detergents, wash additives, etc. In some embodiments, bags 232, 234 may be formed of a plastic material. In some embodiments, the treatment bag 234 may be formed of a fabric 236, such as a mesh material. The fabric 236 of treatment bag 234 may be a soft, recyclable fabric bag, e.g., that is pleasant to touch and handle. Fabric 236 can be pre-washed in a scented composition and then loaded into the interior volume.

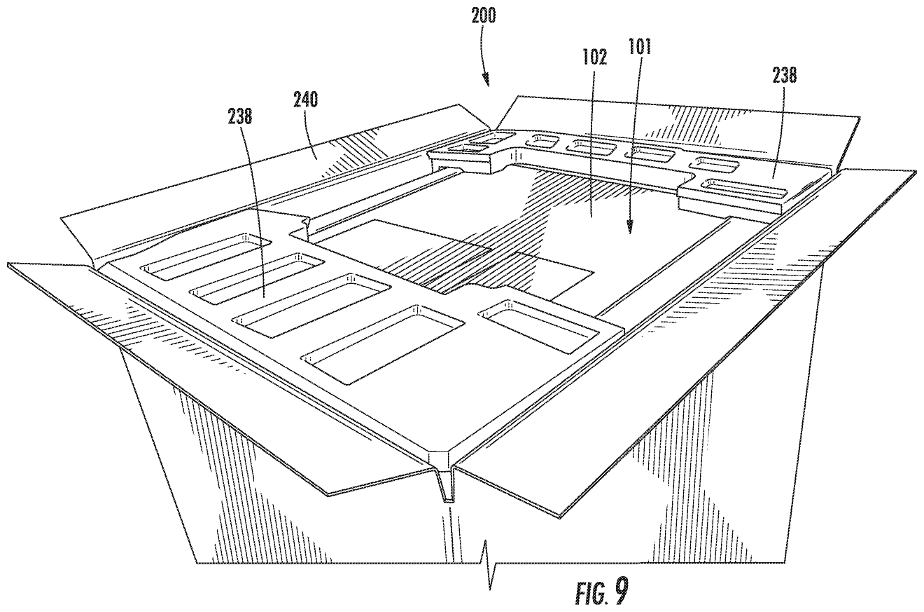

FIG. 9 provides a perspective view of an appliance 101 packaged within a box 240 and supported by EPS support members 238 according to an exemplary embodiment of the present disclosure. For this embodiment, appliance 101 is a horizontal axis washing machine appliance. However, appliance 100 could be any suitable appliance. As shown in FIG. 9, EPS support members 238 are positioned at a top portion of housing 102 of appliance 101, e.g., to support appliance 101 during shipping. Although not shown, EPS support members 238 are likewise positioned at a bottom of housing 102 of appliance 101. For this embodiment, each EPS support member 238 is a scented packaging material 200. Further, each EPS support member 238 is formulated with a scented composition that emanates the preselected scent S. Box 240 is likewise one of the scented packaged materials 200 in this embodiment. Box 240 is formulated with a scented composition that emanates the preselected scent S. Accordingly, as a consumer unloads appliance 101 from box 240 and removes EPS support members 238 from appliance 101, the preselected scent S emanating from box 240 and EPS support members 238 of the scented packaging materials 200 provides the appliance with an attractive smell, which a consumer may smell upon delivery of the appliance.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. An appliance, comprising:
a housing defining an interior volume;
a door coupled with the housing for selectively providing access to the interior volume of the appliance; and
a scented packaging material removably attached to or supporting at least one of the housing and the door, wherein a preselected scent emanates from the scented packaging material.

2. The appliance of claim 1, wherein the scented packaging material is a tape attached to at least one of the housing and the door, the tape comprising a cover layer and an adhesive layer, and wherein the preselected scent emanates from the adhesive layer only upon removal of the tape from the appliance.

3. The appliance of claim 1, wherein the scented packaging material is a tape attached to at least one of the housing and the door, the tape comprising a cover layer and an adhesive layer, and wherein the cover layer comprises one or more porous sections that allow the preselected scent to emanate therethrough.

4. The appliance of claim 3, wherein the adhesive layer comprises boundary walls partitioning adhesive sections, and wherein at least one of the adhesive sections is in fluid communication with one of the one or more porous sections to allow the preselected scent to emanate therethrough even when the tape is attached to at least one of the housing and the door and at least one of the adhesive sections sealed by a non-porous section of the cover layer, the boundary walls, and at least one of the housing and the door when the tape is attached to at least one of the housing and the door.

5. The appliance of claim 1, wherein the scented packaging material is a plastic sheet attached to at least one of the housing and the door, the plastic sheet comprising a protective film and an adhesive layer, and wherein the preselected scent emanates from the adhesive layer only upon removal of the plastic sheet from the appliance.

6. The appliance of claim 1, wherein the door is movable between an open position and a closed position for selectively accessing the interior volume, and wherein the scented packaging material attached to or supporting at least one of the housing and the door is removably disposed within the interior volume, and wherein when the door is moved to the open position, the preselected scent emanating from the scented packaging material emanates from the interior volume.

7. The appliance of claim 1, wherein the preselected scent is perceptible to a human having a normal sense of smell.

8. The appliance of claim 1, wherein the preselected scent is configured to target a specific olfactory gland of a human.

9. The appliance of claim 1, wherein the appliance is a washing machine appliance comprising a tub positioned within the housing and a basket rotatably mounted within the tub, and wherein the scented packaging material is removably disposed within the interior volume and is an expanded polystyrene material positioned to secure the basket in place.

10. The appliance of claim 1, wherein the scented packaging material removably attached to or supporting at least one of the housing and the door is a box in which the appliance is contained.

11. The appliance of claim 1, wherein the scented packaging material removably attached to or supporting at least one of the housing and the door is an expanded polystyrene support member surrounding at least a portion of the housing.

12. The appliance of claim 1, wherein the scented packaging material removably attached to or supporting at least one of the housing and the door is a bag that is removably disposed within the interior volume of the housing.

13. The appliance of claim 12, wherein the bag is formed of a fabric.

14. An appliance, comprising:
a housing defining an interior volume;
a door coupled with the housing and movable between an open position and a closed position for selectively providing access to the interior volume of the appliance; and
one or more scented packaging materials removably attached to or supporting at least one of the housing and the door, wherein a preselected scent emanates from the one or more scented packaging materials, and wherein at least one of the one or more scented packaging materials is removably disposed within the interior volume of the housing, and wherein when the door is moved to the open position, the preselected scent emanating from the scented packaging materials removably disposed within the interior volume emanates to an ambient environment surrounding the appliance.

15. The appliance of claim 14, wherein the at least one of the one or more scented packaging materials that is removably disposed within the interior volume is an expanded polystyrene material.

16. The appliance of claim 14, wherein the at least one of the one or more scented packaging materials that is removably disposed within the interior volume is a bag, and wherein the bag is formed of a fabric, and wherein the fabric is a mesh material.

17. The appliance of claim 14, wherein at least one of the one or more scented packaging materials is a tape attached to at least one of the housing and the door, the tape comprising a cover layer and an adhesive layer, and wherein the preselected scent emanates from the adhesive layer only upon removal of the tape from the appliance.

18. The appliance of claim 14, wherein at least one of the one or more scented packaging materials is a plastic sheet attached to at least one of the housing and the door, the plastic sheet comprising a protective film and an adhesive layer, and wherein the preselected scent emanates from the adhesive layer only upon removal of the plastic sheet from the appliance.
